(19) United States
(12) Patent Application Publication
(10) Pub. No.: US 2014/0243282 A1
(43) Pub. Date: Aug. 28, 2014

(54) METHODS AND COMPOSITIONS FOR DESIGNING NOVEL CONJUGATE THERAPEUTICS

(71) Applicant: Satish Reddy Kallam, (US)

(72) Inventors: Venkateswarlu Akella, Hyderabad (IN); Uday Saxena, Atlanta, GA (US); Anji Reddy, Hyderabad (IN)

(21) Appl. No.: 13/977,271

(22) PCT Filed: Dec. 12, 2011
(86) PCT No.: PCT/IB11/55598

§ 371 Date: May 7, 2014

Related U.S. Application Data
(60) Provisional application No. 61/460,342, filed on Dec. 31, 2010.

(51) Int. Cl. A61K 47/48

(52) U.S. Cl. CPC A61K 47/48038
USPC 514/43; 544/276; 514/263.37; 548/180; 514/367; 536/22.1; 548/339.1; 562/559; 562/575; 562/443; 562/567; 562/562; 548/496; 562/560; 562/557; 562/571; 562/563; 562/104; 548/535

(57) ABSTRACT

The present invention relates to novel drug conjugates, where in two drugs are linked together through an appropriate linker having at least two functional groups capable of forming covalent bond with drugs D1 and D2. The invention also relates to developing novel compositions, methods for their preparation and their use in treating various disease conditions.

### RELATED APPLICATIONS

This application claims priority from U.S. Provisional Patent Application No. 61/460,342 filed 31 Dec. 2010 and is incorporated herein by reference in its entirety.

### BACKGROUND OF THE INVENTION

The field of drug discovery involves interplay of science and technology. The pharmaceutical companies world over are experiencing hard times with the research and development costs spiraling out of control and new drug approvals becoming increasingly scarce. A number of blockbuster drugs are coming off patent and generic competition is intensifying. Both public and investor confidence in the industry have fallen drastically owing to rising drug prices, product safety concerns and late-stage clinical failures. Most of the pharmaceutical innovations are incremental improvements in nature but add up to substantial progress over time. Incremental innovations in drug discovery are seen in various forms including small modifications to pharmacophores in the me-too drug discovery approach, new indications for existing drugs, novel formulations, new dosage forms with improved pharmacokinetic profiles, novel drug delivery techniques etc. Combination therapy with two or more biologically active compounds (drugs) having complementary pharmacological mechanism of action also represents a type of incremental innovation that has extended the range of therapeutic options in the treatment of many diseases. Combination of products also known as fixed dose combination of two or more active drug products in a single tablet has been in practice. These may provide advantages like enhanced activity, lower dosage, reducing cost, minimizing generic competition and extending lifecycle of the product.

All drugs have unwanted side effects in addition to the desired therapeutic effects. The idea of combining two or more drugs with complementary modes of actions is to derive advantages of the synergism or additivity of the desired therapeutic effect. For example the conceptual basis for combination of anti-bacterials is loss of efficacy over a period of time due to the emergence of drug resistant bacterial strains. Some drugs work better in combination such as Augmentin, which is a combination of Amoxicillin and Clavulanic acid. Another case in point is the widely used Septran or Bactrim which is the synergistic combination of sulfamethoxazole and trimethoprim. Such synergistic combinations can be prescribed at lower doses, which could result in lowering side effects and reducing costs.

Thus, drug combination based strategies permit development of improved therapies. In particular there is still a need for technology platforms for developing novel therapies containing two structurally compatible chemical classes of drugs with complementary pharmacological mechanism of actions that provide i) improved pharmacokinetic properties, ii) better control over disease conditions iii) reduced pathological risk factors iv) improved treatment options v) lower side effect profile over individual drugs.

Most technologies available today to improve pharmacokinetic properties of drugs are applicable to use of single drugs or mono-therapy. However, combination drug therapy especially combining two different drugs has become necessary and increasingly common to treat a plethora of modern day diseases. Yet, there are no good methods to improve pharmacokinetic properties and efficacy properties of drugs when used as combination therapy. Even incremental improvements in pharmacokinetic properties and efficacy of a drug combination could have enormous benefits to the patients. Currently, when combination drugs are used, the drug components are simply mixed together as a physical mixture which has significant issues with regards to optimal pharmacokinetic properties, efficacy and formulation.

None of the available art discloses or suggests prodrug conjugates of two or more of drugs linked to one another through amino acid linker. Nor do they disclose drug conjugates which are linked by reversible covalent bonds, so that at the desired site in the body they are cleaved to regenerate the active forms of each of the drugs.

There is a need in the pharmaceutical arts for pharmaceutical compounds which deliver two or more drugs at a single time in a single dose, which exhibit controlled drug delivery. The drug conjugates of the present invention would expect to confer the advantage that linking the two drug compounds alters the solubility and pharmacokinetic properties of each through the covalent bonds linking the compounds. The drug conjugates of the invention have a high degree of enzymatic susceptibility at physiological pH 7.4.

The single chemical entity of an amino acid linked drug conjugate is expected to result in superior pharmacokinetic properties and translate into improved therapeutic effects compared to a physical mixture of the same drugs. Accordingly, the present invention envisages that the desired functional groups of two drugs are covalently bonded with the amino acid linker to produce a single new chemical entity, as a novel therapeutic for treatment of diseases.

### SUMMARY OF INVENTION

The present invention relates to novel drug conjugates of formula (I), where in two drugs are linked together through an appropriate linker having at least two functional groups capable of forming covalent bond with drugs D1 and D2

The present invention comprises a novel class of drug conjugates of formula (I), that are therapeutically useful, and methods of preparing these compounds. Accordingly, the compounds and pharmaceutical compositions of this invention are useful in the treatment and prevention of variety of diseases.

In general, the conjugates of formula (I) can be represented by

Wherein ‘D1’ is Drug-1, which contains one or more functional groups selected from the groups comprising of —OH, —COOH, —SH, —NHR1, —CONH—R1, —SO2NH—R1, —OSO2NH—R1, —NHR1-CO—NHR1, —NR1SO2NH—R1;

R1 represents hydrogen, —NH2, —C(═NH)NH2, alkyl, aryl, heteroaryl, or heterocyclyl;

‘D2’ represents ‘Drug-2’, is selected from a therapeutic, a vitamin, a natural product or a neutraceutical; D2 also contains one or more functional groups as defined above for drug one;

Linker is selected from

i) an amino acid AA

Wherein amino acid AA comprising of two or more functional groups, such as carboxyl, hydroxyl, amino, mercapto or guanidine groups;

Amino acid may be chosen from L-form, D-form or DL-forms;

AA also includes formulas represented by:

(a)

wherein R2 is a group selected from (CH2)n-COOH, —CH3, —(CH2)n-NH—C(═NH)—NH2, —CH2SH, —(CH2)nCONH2, —(CH2)n-NH2, —(CH2)nCH2-alkyl, —(CH2)nCH2-heterocylyl, —(CH2)nCH2-heteroaryl, —(CH2)nCH2-aryl, —(CH2)nCH2-phenyl, or —(CH2)nCH2-p-hydroxy phenyl; R2 forms cyclic ring with NH2 as in praline;

(b)NH2-(CH2)n-COOH;

ii) Hydroxy acids represented by

—O—CH(R1)-(CH2)n-COO—

—O—CH(R1)-(CH2)n-CH(R1)-COO—

—O—(CH2)n-CH(R1)-COO—

—OOC—CH(R1)-(CH2)n-O—

—OOC—CH(R1)-(CH2)n-CH(R1)-O—

—OOC—(CH2)n-CH(R1)-O—

—OOC—CH(R1)-(CH2)n-O—

—OOC—(CH2)n-CH(R1) (CH2)n-O—;

—O—Ar—CH(R1)-(CH2)n-COO—

—O—Ar—CH(R1)-(CH2)n-CH(R1)-COO—

—O—(CH2)n-Ar—CH(R1)-COO—

—OOC—CH(R1)-Ar—(CH2)n-O—

iii) Mercapto carboxylic acids represented by

—S—CH(R1)-(CH2)n-COO—

—S—CH(R1)-(CH2)n-CH(R1)COO—

—S—(CH2)n-CH(R1)-COO—

—S—(CH2)n-CH(R1)(CH2)n-COO—

—S—Ar—CH(R1)-(CH2)n-O—

—S—CH(R1)-(CH2)n-CH(R1)-Ar—O—

—S—(CH2)n-Ar—CH(R1)-O—

—S—(CH2)n-CH(R1)-Ar—(CH2)n-O—

iv) Hydroxy mercapto linkers represented by

—O—CH(R1)-(CH2)n-S—

—O—CH(R1)-(CH2)n-CH(R1)-S—

—O—(CH2)n-CH(R1)-S—

—O—(CH2)n-CH(R1)(CH2)n-S—

v) Mercapto hydroxy linkers represented by

—S—CH(R1)-(CH2)n-O—

—S—CH(R1)-(CH2)n-CH(R1)-O—

—S—(CH2)n-CH(R1)-O—

—S—(CH2)n-CH(R1)(CH2)n-O—

vi) Diamino linkers represented by

—NH2-Ar—CH(R1)-(CH2)n-NH2-

—NH2-Ar—CH(R1)-(CH2)n-NH2-

—NH2-CH(R1)-Ar—(CH2)n-CH(R1)-NH2-

—NH2-(CH2)n-CH(R1)-Ar—NH2-

Vii) Dicarboxylic acid linkers represented by

—OOC—CH(R1)-(CH2)n-COO—

—OOC—CH(R1)-(CH2)n-CH(R1)-COO—

—OOC—(CH2)n-CH(R1)-COO—

—O—CH(R1)-(CH2)n-COO—

—OOC—Ar—CH(R1)-(CH2)n-COO—

—OOC—CH(R1)-Ar—(CH2)n-CH(R1)-COO—

Viii) Carboxylicacid mercapto linkers of type

—OOC—(CH2)n-CH(R1)(CH2)n-S—

—O—CO—CH(R1)-(CH2)n-CH(R1)-S—

—O—CO—(CH2)n-CH(R1)-S—

—O—CO—(CH2)n-CH(R1)(CH2)n-S—

—OOC—Ar—(CH2)n-CH(R1)(CH2)n-S—

—O—CO—CH(R1)-(CH2)n-CH(R1)-AR-S—

—O—CO—(CH2)n-Ar—CH(R1)-S—

—O—CO—(CH2)n-CH(R1)-Ar—(CH2)n-S—

ix) Hydroxy amino linkers represented by

—O—CH(R1)-(CH2)n-NH—

—O—CH(R1)-(CH2)n-CH(R1)-NH—

—O—(CH2)n-CH(R1)-NH—

—O—(CH2)n-CH(R1)(CH2)n-NH—

—OAr—CH(R1)-(CH2)n-NH—

—O—CH(R1)-(CH2)n-CH(R1)-Ar—NH—

—O—(CH2)n-Ar—CH(R1)-NH—

—O—(CH2)n-CH(R1)-Ar—(CH2)n-NH—

x) Amino hydroxy linker

—NH—CH(R1)-(CH2)n-OH—

—NH—CH(R1)-(CH2)n-CH(R1)-O—

—NH—(CH2)n-CH(R1)-O—

—NH—(CH2)n-CH(R1)(CH2)n-O—

—NH—CH(R1)-Ar—(CH2)n-OH—

—NH—CH(R1)-(CH2)n-Ar—CH(R1)-O—

—NH—(CH2)n-CH(R1)-Ar—O—

—NH—Ar—(CH2)n-CH(R1)(CH2)n-O—

n is an integer from 0-6;

D1, D2 each independently represents drug of either same therapeutic class or of different therapeutic class; when they represent same therapeutic class each one may be selected from drugs of pharmacologically different mechanism of actions;

D1, D2 are independently represent therapeutically compatible drugs, each independently covalently linked to the two different functional groups of AA linker; AA linker allows the release of D1 or D2 or both in the internal or external environment of the target tissue; D1, D2 may be released either simultaneously or one after the other at different time points, depending on the nature of drug and covalent bond between drug and AA linker and environment of target tissue;

D1 and D2 each independently covalently bonded to Linker through one or more of the following:

i) —O—

ii) —C(O)—O—

iii) —NH—

iv) —NH—C(O)—

v) —O—C(O)—

vi) —S—

vii) —C(O)—NH—

in a more specific embodiment, D1 and D2 each independently is covalently linked either to —NH2 functional group or —COOH functional group of AA;

D1 or D2 is covalently bonded to AA through

i) —O—C(O)—

ii) —NH—C(O)—

iii) —C(O)NH—

iv) —C(O)—O bonds

The present invention relates to novel drug conjugates, wherein the desired functional groups of two drugs are linked through an amino acid linker with covalent bonding, with intent of improving pharmacokinetic properties and efficacy.

The present invention also uses in silico, in vitro and in vivo methods to select the suitable amino acid as linker for combining two drugs to produce a single chemical entity, which is a prodrug.

Another object of the present invention describes a conjugation of two drugs, which are linked through enzymatically labile bonds with amino acid linker moiety comprising two linker functional groups. The suitable functional group of Drug-I is covalently bonded to the desired functional group at the first end of the amino acid linker moiety and the suitable functional group of Drug-II is covalently bonded to the desired functional group at the second end of the linker moiety, wherein the drug conjugate will be cleaved to regenerate the active forms of the drug compounds at the required sites in the body.

Another object of the present invention describes pharmaceutical compositions comprising a prodrug of the drug conjugates.

Yet another object of the present invention provides pharmaceutical compositions comprising a prodrug of the present invention for the treatment of various disease conditions, specific to the individual drugs of the drug conjugates.

### DETAILED DESCRIPTION OF INVENTION

### Definitions

As used herein, the term “alkyl,” is intended to include both branched and straight-chain saturated aliphatic hydrocarbon groups having a specified number of carbon atoms. Exemplary alkyl groups of the invention have from 1 to 10 carbon atoms. Branched means a lower alkyl group such as methyl, ethyl or propyl, is attached to a linear alkyl chain. Non-limiting examples of suitable alkyl groups include methyl, ethyl, n-propyl, isopropyl, n-butyl, and t-butyl.

As used herein, the term “aryl” means an aromatic or partially aromatic monocyclic or polycyclic ring system comprising about 6 to about 14 carbon atoms, preferably about 6 to about 10 carbon atoms. Non-limiting examples of suitable aryl groups include phenyl, naphthyl, 1, 2,3,4-tetrahydro-naphthyl, and indanyl.

As used herein, the term “heteroaryl” means an aromatic monocyclic or polycyclic ring system of 4 to 10 carbon atoms, having at least one heteroatom selected from —O—, >N— or —S—. Exemplary heteroaryl groups include as pyrazinyl, isothiazolyl, oxazolyl, pyrazolyl, pyrrolyl, triazolyl, tetrazolyl, oxatriazolyl, oxadiazolyl, pyridazinyl, thienopyrimidyl, furanyl, indolyl, isoindolyl, benzo[1,3]dioxolyl, 1,3-benzoxathiole, quinazolinyl, isoquinolinyl, quinolinyl, pyridyl, 1,2,3,4-tetrahydro-isoquinolinyl, 1,2,3,4-tetrahydro-quinolinyl pyridyl, thiophenyl, and the like.

As used herein, the term “heterocyclyl” means a non-aromatic, saturated or unsaturated, monocyclic or polycyclic ring system of 3 to 10 member having at least one heteroatom selected from —O—, >N— or —S—. Exemplary heterocyclyl groups include aziridinyl, imidazolidinyl, 2,5-dihydro-[1,2,4]oxadiazolenyl, oxazolidinyl, isooxazolidinyl, pyrrolidinyl, piperdinyl, piperazinyl, morpholinyl, thiomorpholinyl, thiazolidinyl, 1,3-dioxolanyl, 1,4-dioxanyl, 2,5-dihydro-1H-imidazolyl, and the like

The term “Drug-I” is intended to encompass any pharmacological agent effective in inhibiting, attenuating, combating or overcoming any disease condition, and contains the functional groups as defined in the description of formula (I) below.

The term “Drug-II” is intended to encompass any pharmacological agent effective in inhibiting, attenuating, combating or overcoming any disease condition, and contains the functional groups as defined in the description of formula (I) below.

Drug conjugates are formed by conjugation of two or more drugs via enzymatically labile bonds. Drug conjugates are linked via reversible covalent bonds so that they are cleaved to regenerate the active forms of the drug compounds at the required site in the body. The rate of cleavage of the two drugs can be controlled by the type of bond, the choice of drugs and the physical form of the conjugate. The drug conjugates are labile in water, serum or other bodily fluids and regenerate the active parent drugs. The present invention is for the first time combines two or more drugs in the form of a drug conjugate through amino acid linker which generates two active drug compounds in vivo, with improved pharmaceutical properties. drug conjugates have the applicability of providing a controlled or sustained release for a systemic or local pharmacologic or physiologic effect relating to the following areas: treatment of cancerous primary tumors; chronic pain; tuberculosis; infectious diseases; cardiovascular diseases; central nervous system (CNS) disorders; neurodegenerative diseases such as Alzheimer's; arthritis; rheumatic conditions; metabolic deficiencies such as diabetes; and modifications of the immune response such as in the prevention of transplant rejection and in cancer therapy. A wide variety of disease states may be prevented or treated using the drug conjugate compositions of the present invention. Such disease states are known to those of ordinary skill in the art (see Goodman and Gilman, The Pharmacological Basis of Therapeutics, 8th Ed., Pergamon Press, NY, 1990; and The Merck Index, 11th Ed., Merck and Co., Inc., Rahway, N.J. 1996; incorporated herein by reference in their entireties)

A “Prodrug” is a compound formed by chemical modification of a biologically active compound which will liberate the active compound in vivo by enzymatic or hydrolytic cleavage. The primary purpose of employing a prodrug is to increase intestinal absorption or site specific absorption or to reduce local side effects, such as gastrointestinal irritation. Prodrugs may also be used to increase transdermal absorption, by enhancing permeation through topical membranes.

As used herein, the term “Amino acid linker” is defined as any amino acid molecule with suitable functional groups, to which desired functional groups of Drug-I and Drug-II are bonded covalently.

The present invention provides a means of improving the pharmaceutical and pharmacological properties of pharmacologically active compounds or prodrugs by conjugating them together to form a drug conjugate.

One aspect of the present invention relates to the compound of formula (I), that are therapeutically useful, and methods of preparing these compounds. Accordingly, the compounds and pharmaceutical compositions of this invention are useful in the treatment and prevention of variety of diseases.

In general, the conjugates of formula (I) can be represented by

Wherein ‘D1’ is Drug-1, which contains one or more functional groups selected from the groups comprising of —OH, —COOH, —SH, —NHR1, —CONH—R1, —SO2NH—R1, —OSO2NH—R1, —NHR1-CO—NHR1, —NR1 SO2NH—R1;

R1 represents hydrogen, —NH2, —C(═NH)NH2, alkyl, aryl, heteroaryl, or heterocyclyl;

‘D2’ represents ‘Drug-2’, is selected from a therapeutic, a vitamin, a natural product or a neutraceutical; D2 also contains one or more functional groups as defined above for drug one;

Linker is selected from

i) an amino acid AA

Wherein amino acid AA comprising of two or more functional groups, such as carboxy, hydroxyl amino, mercapto or guanidine groups;

Amino acid may be chosen from L-form, D-form or DL-forms;

AA also includes formulas represented by

(a)

wherein R2 is a group selected from (CH2)n-COOH, —CH3, —(CH2)n-NH—C(═NH)—NH2, —CH2SH, —(CH2)nCONH2, —(CH2)n-NH2, —(CH2)nCH2-alkyl, —(CH2)nCH2-heterocylyl, —(CH2)nCH2-heteroaryl, —(CH2)nCH2-aryl, —(CH2)nCH2-phenyl, or —(CH2)nCH2-p-hydroxy phenyl; R2 forms cyclic ring with NH2 as in praline;

(b)NH2-(CH2)n-COOH;

ii) Hydroxy acids represented by

—O—CH(R1)-(CH2)n-COO—

—O—CH(R1)-(CH2)n-CH(R1)-COO—

—O—(CH2)n-CH(R1)-COO—

—OOC—CH(R1)-(CH2)n-O—

—OOC—CH(R1)-(CH2)n-CH(R1)-O—

—OOC—(CH2)n-CH(R1)-O—

—OOC—CH(R1)-(CH2)n-O—

—OOC—(CH2)n-CH(R1)(CH2)n-O—;

—O—Ar—CH(R1)-(CH2)n-COO—

—O—Ar—CH(R1)-(CH2)n-CH(R1)-COO—

—O—(CH2)n-Ar—CH(R1)-COO—

—OOC—CH(R1)-Ar—(CH2)n-O—

iii) Mercapto carboxylic acids represented by

—S—CH(R1)-(CH2)n-COO—

—S—CH(R1)-(CH2)n-CH(R1)-COO—

—S—(CH2)n-CH(R1)-COO—

—S—(CH2)n-CH(R1)(CH2)n-COO—

—S—Ar—CH(R1)-(CH2)n-O—

—S—CH(R1)-(CH2)n-CH(R1)-Ar—O—

—S—(CH2)n-Ar—CH(R1)-O—

—S—(CH2)n-CH(R1)-Ar—(CH2)n-O—

Iv) Hydroxy mercapto linkers represented by

—O—CH(R1)-(CH2)n-S—

—O—CH(R1)-(CH2)n-CH(R1)-S—

—O—(CH2)n-CH(R1)-S—

—O—(CH2)n-CH(R1)(CH2)n-S—

v) Mercapto hydroxy linkers represented by

—S—CH(R1)-(CH2)n-O—

—S—CH(R1)-(CH2)n-CH(R1)-O—

—S—(CH2)n-CH(R1)-O—

—S—(CH2)n-CH(R1) (CH2)n-O—

vi) Diamino linkers represented by

—NH2-Ar—CH(R1)-(CH2)n-NH2-

—NH2-Ar—CH(R1)-(CH2)n-NH2-

—NH2-CH(R1)-Ar—(CH2)n-CH(R1)-NH2-

—NH2-(CH2)n-CH(R1)-Ar—NH2-

Vii) Dicarboxylic acid linkers represented by

—OOC—CH(R1)-(CH2)n-COO—

—OOC—CH(R1)-(CH2)n-CH(R1)-COO—

—OOC—(CH2)n-CH(R1)-COO—

—O—CH(R1)-(CH2)n-COO—

—OOC—Ar—CH(R1)-(CH2)n-COO—

—OOC—CH(R1)-Ar—(CH2)n-CH(R1)-COO—

Viii) Carboxylicacid mercapto linkers of type

—OOC—(CH2)n-CH(R1)(CH2)n-S—

—O—CO—CH(R1)-(CH2)n-CH(R1)-S—

—O—CO—(CH2)n-CH(R1)-S—

—O—CO—(CH2)n-CH(R1)(CH2)n-S—

—OOC—Ar—(CH2)n-CH(R1)(CH2)n-S—

—O—CO—CH(R1)-(CH2)n-CH(R1)-AR-S—

—O—CO—(CH2)n-Ar—CH(R1)-S—

—O—CO—(CH2)n-CH(R1)-Ar—(CH2)n-S—

ix) Hydroxy amino linkers represented by

—O—CH(R1)-(CH2)n-NH—

—O—CH(R1)-(CH2)n-CH(R1)-NH—

—O—(CH2)n-CH(R1)-NH—

—O—(CH2)n-CH(R1)(CH2)n-NH—

—OAr—CH(R1)-(CH2)n-NH—

—O—CH(R1)-(CH2)n-CH(R1)-Ar—NH—

—O—(CH2)n-Ar—CH(R1)-NH—

—O—(CH2)n-CH(R1)-Ar—(CH2) n-NH—

x) Amino hydroxy linkers of type

—NH—CH(R1)-(CH2)n-OH—

—NH—CH(R1)-(CH2)n-CH(R1)-O—

—NH—(CH2)n-CH(R1)-O—

—NH—(CH2)n-CH(R1)(CH2)n-O—

—NH—CH(R1)-Ar—(CH2)n-OH—

—NH—CH(R1)-(CH2)n-Ar—CH(R1)-O—

—NH—(CH2)n-CH(R1)-Ar—O—

—NH—Ar—(CH2)n-CH(R1)(CH2)n-O—

n is an integer from 0-6;

D1, D2 each independently represents drug of either same therapeutic class or of different therapeutic class; when they represent same therapeutic class each one may be selected from drugs of pharmacologically different mechanism of actions;

D1, D2 are independently represent therapeutically compatible drugs, each independently covalently linked to the two different functional groups of AA linker; AA linker allows the release of D1 or D2 or both in the internal or external environment of the target tissue; D1, D2 may be released either simultaneously or one after the other at different time points, depending on the nature of drug and covalent bond between drug and AA linker and environment of target tissue;

D1 and D2 each independently covalently bonded to Linker through one or more of the following:

i) —O—

ii) —C(O)—O—

iii) —NH—

iv) —NH—C(O)—

v) —O—C(O)—

vi) —S—

vii) —C(O)—NH—

In a more specific embodiment, D1 and D2 each independently is covalently linked either to —NH2 functional group or —COOH functional group of AA;

D1 or D2 is covalently bonded to AA through

i) —O—C(O)—

ii) —NH—C(O)—

iii) —C(O)NH—

iv) —C(O)—O bonds

AA represents an amino acid, which acts as linker between Drug-I and Drug-II, includes but not restricted to those provided in the below Table 1:

```
TABLE 1
Exemplary Amino acids of the invention
  L-Histidine
  L-Methionine
  L-Isoleucine
  L-Phenylalanine
  L-Leucine
  L-Threonine
  L-Lysine
  L-Tryptophan
  L-Valine
  L-Alanine
  L-Arginine
  L-Cystine
  L-Aspartic acid
  L-Glutamic acids
  L-Cysteine
  L-Glutamine
  Glycine
  L-Serine
  L-Ornithine
  Taurine
  L-Proline
  L-Tyrosine
  Beta-Aminopropionic acid
  Gamma-Aminobutyric acid
```

or its stereo isomers thereof.

In one aspect D1 or D2 is covalently bonded to AA through

i) —O—C(O)—

ii) —NH—C(O)—

iii) —C(O)NH—

iv) —C(O)—O bonds

In one more aspect the exemplary categories of novel conjugates of this invention are illustrated below.

Wherein FG is a functional group, D1 is —OH containing Drug-1, D2- is —COOH containing Drug-2; AA is an amino acid of formula

—NH2 functional group of AA is covalently bonded to —COOH functional group of D2 through —NH—CO-bond; —OH containing D1 form a covalent bond with HO—CO—CH(R2)-NH—CO-D2 residue to form compounds of formula (I)

Wherein FG is a functional group, D1 is —NH2 containing Drug-1, D2- is —COOH containing Drug-2; AA is an amino acid of formula

—NH2 functional group of AA is covalently bonded to —COOH functional group of D2 through —NH—CO-bond; —NH2 containing D1 form a covalent bond with HO—CO—CH(R2)-NH—CO-D2 residue to form compounds of formula (I)

Wherein FG is a functional group, D1 is —COOH containing Drug-1, D2- is —NH2 containing Drug-2; AA is an amino acid of formula

—NH2 functional group of AA is covalently bonded to —COOH functional group of D1 through —CO—NH-bond; —NH2 containing D2 form a covalent bond with D1-CO—NH—CH(R2)-CO—OH residue to form compounds of formula (I)

Wherein FG is a functional group, D1 is —COOH containing Drug-1, D2- is —OH containing Drug-2; AA is an amino acid of formula

—NH2 functional group of AA is covalently bonded to —COOH functional group of D1 through —CO—NH bond; —OH containing D2 form a covalent bond with D1-CO—NH—CH(R2)-COOH residue to form compounds of formula (I)

Wherein FG is a functional group, D1 is —COOH containing Drug-1, D2- is also COOH containing Drug; AA is an amino acid of formula

where in R2 is —COOH, —(CH)n-COOH and n iso-5; —NH2 functional group of AA is covalently bonded to —COOH functional group of D1 through —CO—NH bond; —OH containing D2 form a covalent bond with D1-CO—NH—(CH2)n CH(R2)-NH2 residue to form compounds of formula (I)

Wherein FG is a functional group, D1 is —OH containing Drug-1, D2- is also —OH containing Drug; AA is an amino acid of formula

wherein R2 is —NH2, and n iso-5; —COOH functional group of AA is covalently bonded to —OH functional group of D1 through —OCO bond; —OH containing D2 form a covalent bond with D1-O—CO—(CH2)n CH(R2)COOH residue to form compounds of formula (I)

Wherein FG is a functional group, D1 is —NH2 containing Drug-1, D2- is also —NH2 containing Drug; AA is an amino acid of formula

wherein R2 is —NH2, and n iso-5; —COOH functional group of AA is covalently bonded to —NH2 functional group of D1 through —NHCO bond; —NH2 containing D2 form a covalent bond with D1-NH—CO—(CH2)n CH(R2)COOH residue to form compounds of formula (I)

Wherein FG is a functional group, FG is a functional group, D1 is —NH2 containing Drug-1, D2 is —OH2 containing Drug; AA is an amino acid of formula

wherein R2 is —NH2, and n iso-5; —COOH functional group of AA is covalently bonded to —NH2 functional group of D1 through —NHCO bond; —NH2 containing D2 form a covalent bond with D1-NH—CO—(CH2)n CH(R2)COOH residue to form compounds of formula (I)

Wherein FG is a functional group, D1 is —NH2 containing Drug-1, D2 is —OH2 containing Drug; AA is an amino acid of formula

wherein n is 0-5; —COOH functional group of AA is covalently bonded to —NH2 functional group of D1 through —NHCO bond; —OH containing D2 form a covalent bond with D1-NH—CO—(CH2)n COOH residue to form compounds of formula (I)

In one aspect the invention is further illustrated by the following compounds of formula (I)

And their stereo isomers

In compounds of formulae above,

In one aspect, D1, D2 independently selected from different therapeutic classes of drugs like analgesic/antipyretic drugs, antihypertensive drugs, antibiotics, antibacterial drugs, antifungal drugs, antiviral drugs, antimalarial drugs, antineoplastic drugs, antioxidants/free radical scavengers, tranquilizers and hypnotics, antiulcer drugs, anticonvulsants, antiparkinsons drugs, antidepressants, antihistaminic drugs, anti-inflammatory drugs, cardioprotective drugs, antidiabetic drugs, drugs for respiratory diseases, cardiovascular drugs, gastro intestinal drugs, drugs for skin diseases, drugs for genito urinary diseases, drugs of musculo-skeletal disorders, rugs of endocrine system, drugs of allergy, anaesthetics;

In one more aspect the following pairs of drugs combined with AA linker are exemplified hereunder as drugs for combination therapy

Two drugs with in the anticancer class of drugs, two drugs of with in the antibacterials, antilpidemic and hypertension, antidiabetic and hypertension, antidiabetic and antidiabetic, antiasthmatic and COPD, anti-inflammation, arthritis and ulcers, antiviral and antiviral

Another aspect of the present invention relates to the development of novel drug combination platform technology suitable for developing novel compositions comprising of the following steps:

Identifying two known drugs or compounds having potential to become therapeutic agents,

Evaluation of amino acid linkers to identify the suitable linker to combine the drugs or compounds identified in step (i),

combining the two drugs or compounds through covalent bonds with the linker identified in step (ii), to obtain a novel compound of formula (I),

in vitro evaluation of novel compounds formed in step (iii) for intended therapeutic activity,

Determining pharmacokinetic experiments in vitro/in vivo to asses the cleavability and to evaluate the release kinetics of combined drugs and

Determining in-vivo efficacy of combined drugs/novel compounds of formula (I) of step (iii).

Various steps involved in identifying and determining the novel combination are elaborated below:

(i) Identifying suitable drugs or compounds for combination: known drugs or compounds under various therapeutic categories can be identified,

(ii) In silico selection of amino acid linker: The selection of amino acid will be rational based on two criteria: a) the two drugs to be combined and b) the therapeutic area that the drugs will be used for. A careful in silico analysis of the functional groups available for linking to an amino acid will be conducted. The analysis will include functional groups available for coupling to an amino or carboxylic group in the amino acid, the role and effects of this functional group in the pharmacophore and biological activity of the drugs, potential improvements in solubility by using a positively charged, negatively charged or neutral amino acid. Secondly, the amino acid to be used will also be evaluated for its potential to add to any therapeutic effects of the two drugs. For example the use of arginine to link two cardiovascular drugs will be important because arginine is a precursor of nitric oxide, a vasodilator with several beneficial effects for cardiovascular disease. Another such example is the use of hydroxyisoleucine to combine anti-diabetic drugs, since this amino acid has been shown to increase insulin secretion. Tryptophan is a precursor for serotonin, a neurotransmitter and therefore of therapeutic value to link drugs for CNS diseases.

(iii) Assessment of cleavability and release kinetics of amino acid linked drugs as a measure of improved in vivo pharmacokinetic properties: The amino acid linked drugs will be synthesized and then in order to test the cleavability and release kinetics under physiological conditions they will be subjected to incubation in human plasma. Human plasma is a rich source of a variety of physiological esterases, peptidases and proteases. Thus any such cleaving enzyme that the drug combination is likely to encounter should be mimicked by plasma incubation. Also since the plasma is a rich source of proteins (mainly albumin) this protocol is likely to reflect any impact of protein binding to its suitability to cleavage. Protein binding is a dominant part of the drugs metabolic fate in vivo and can make or break the drugs efficacy profile. Once incubated with plasma, the whole mix will be subjected to LC-MS to be able to quantitate the intact drug combination and the cleaved products.

(iv) In vitro activity of drug combination: Once the drug combination is found to have the desirable cleavage pattern and release kinetics, the drug combination will be subjected to in vitro screening of compounds. The objective of this screen would be to demonstrate that the amino acid linked drug combination is cleaved and then is biologically active. The screens will be conducted with cells incubated in serum/plasma containing conditions to allow for cleavage as well as uptake of the released drugs by the cells. This screening will provide an early read-out of the cleavability of the amino acid linked drugs as well as the ability of the drugs to affect cellular processes resulting in therapeutic activity under in vivo like conditions.

(v) In vivo pharmacokinetic analysis of combination: The pharmacokinetics of the test compounds are done in a suitable animal species like rat, mice or dog and different parameters like Cmax, Tmax, AUC 0-24, AUC 0 to infinity are determined at appropriate doses and compared with individual drug molecules with linker as per the reported protocols.

(vi) Efficacy assessment of combination: Depending on the therapeutic category of drugs identified, in vivo efficacy of the drugs will be evaluated at appropriate doses as per the published protocols in disease specific models. For example conventional models like Ob/Ob mice, Db/Db mice, kkAy mice, can be used as diabetes models. Similarly Diet Induced Obese mice, Zucker fa/fa rats for obesity, collagen induced arthritis mice or rats, and adjuvant induced arthritis rats for arthritis.

### Methods of Preparation

The compounds of this invention may be synthesized as shown in scheme I-III

The compounds described herein may be prepared by techniques known in the art. In addition, the compounds described herein may be prepared by following the reaction sequence as depicted in Scheme-I-III. Further, in the following schemes, where specific bases, acids, reagents, solvents, coupling agents, etc., are mentioned, it is understood that other bases, acids, reagents, solvents, coupling agents etc., known in the art may also be used and are therefore included within the present invention. Variations in reaction conditions, for example, temperature and/or duration of the reaction, which may be used as known in the art, are also within the scope of the present invention. All the stereo isomers of the compounds in these schemes, unless otherwise specified, are also encompassed within the scope of this invention

The compounds of the present invention may for example, be synthesized according to a general process as illustrated below.

Briefly, D1 and D2 can be covalently linked together with an appropriate linker by well known coupling techniques. D1 and D2 may be linked together in any order as long as the final compound corresponds to compounds of the present invention. For example D1 can be linked to D2-linker; or D2 can be linked to D1-linker

When linker is an amino acid, coupling is between D1 or D2, coupling can be carried out using standard coupling procedures such as azide method, mixed carbonic-carboxylic acid anhydride (isobutyl chloroformate) method carbodiimide (dicyclohexyl carbodiimide, diisopropyl crabodiimide or water soluble carbodiimide) method, active ester (p-nitro phenyl ester, N-hydroxy succinic imido ester) method, carbonyl imidazole methods. Some of these methods can be improved by adding 1-hydroxybenzotriazole or 4-DMAP. These coupling reactions can be performed in ether solution or solid phase.

More explicitly, the coupling step involves the dehydrative coupling of free carboxyl of one reactant in the presence of a coupling agent to form a linking amide bond. Descriptions of such coupling agents are found in general text books on peptide chemistry. Examples of suitable coupling agents are N,N′-dicyclohexylcarbodiimide, 1-hydroxybenzotriazole, in the presence of N-ethyl-N′[(3-dimethylamino)propyl carbodiimide]. A practical and useful coupling agent is hexa fluorophosphates reagents. The coupling reaction is conducted in an inert solvent like dichloromethane, acetonitrile or DMF. An excess of tertiary amine such as d isopropylamine, n-methyl morpholine, n-methylpyrroloidine or DMAP is added to maintain Ph of about 8. The reaction temperature may range from 0-50 C and the reaction time between 15 min and 24 hours.

The other functional groups present in the reactants must be protected during the coupling reactions to avoid formation of undesired bonds. Protecting groups are listed for example I in Grene, “Protective groups in organic chemistry” which is hereby incorporated by reference. Examples of protecting groups include, acyl groups, carbamates, alkyl groups, trialkyl silyl and thiol containing groups.

### Scheme-I:

A further aspect of the present invention relates to a process for preparing compounds of formula (I) comprising

i. reacting the carboxyl containing Drug-I or compound with an amino group of amino acid linker, in its carboxy group protected form, in the presence of a suitable coupling agent, a base and a solvent.

wherein Y¹ represents OH or carboxyl protecting group such as methoxy, ethoxy and the like.

R₂ represents a functional group specific to the Amino acid linker used.

Alternatively, the carboxyl containing drug or compound can be first converted to reactive carbonyl derivative such as an acid halide,

wherein Y¹ and R₂ are as defined in step (i).

ii. deprotecting the carboxylic group of the Drug-I or compound obtained in the above step i, in the presence of a suitable base and a solvent to afford the corresponding acid.

wherein Y¹ and R₂ are as defined in step (i).

iii. reacting the carboxylic acid obtained in the above step ii with hydroxyl containing Drug-II or compound in the presence of a suitable base and a solvent to afford compounds of formula (Ia)

### Scheme-II:

According to another aspect of the invention, compounds of formula (Ia) are prepared by,

i. reacting the amino group containing Drug-I or compound with the carboxylic acid group of amino acid linker, in its amino protected form, in the presence of a suitable coupling agent, a base and a solvent or by activating carboxyl group of amino acid by a leaving group and then reacting with Drug-I,

wherein Y represents an amino protecting group such as tert-butoxylcarbonyl (tert-BOC) and the like. LG represents a leaving group such as methoxy, ethoxy and the like;

ii. deprotecting the compound obtained in the above step i, by using suitable conditions known in the art, to afford the corresponding amine,

D1-NH—CO—CHR₂—NHY→D1-NH—CO—CHR₂—NH₂

wherein Y is as defined in step i above;

iii. reacting the amine obtained in the above step ii with a carboxyl containing Drug-II or compound in the presence of a suitable coupling agent, a base and a solvent to afford compound of formula (Ib),

### Scheme-III:

According to another aspect of the invention, compounds of formula (Ib) are prepared by,

i. reacting the amino containing Drug-I or compound with an acid group of amino acid linker, in its amino protected form, in the presence of a suitable coupling agent, a base and a solvent or by activating carboxyl group of amino acid by a leaving group and then reacting with Drug-I

wherein Y represents an amino protecting group tert-butyloxy carbonyl (t-BOC) and the like.

Y¹ represents a protecting or a leaving group on carboxylic function such as methoxy, ethoxy and the like.

ii. deprotection of the second carboxyl function of compound obtained in the above step i, by known methods

wherein Y represents amino protecting group such as tert-butyloxy carbonyl (t-BOC) and the like.

Y¹ represents a protecting or leaving group on carboxylic function such as methoxy, ethoxy and the like.

iii. reacting the compound obtained in the above step ii with —NH₂ containing Drug-2.

One more aspect of this invention describes the compounds of formula I, by combining two acid containing drugs with an amino acid containing two —NH₂ functional groups.

One more aspect of this invention is the compound of formula (I), wherein Drug-I and Drug-II represent different therapeutic agents of same or different therapeutic class. These therapeutic drugs may have same or different pharmacological mechanism of actions or work on different disease conditions.

A further aspect of the present invention relates to pharmaceutical compositions comprising a therapeutically effective amount of the compound of formula (I), or a pharmaceutical salt thereof and one or more pharmaceutically acceptable carriers, vehicles or diluents.

A further aspect of present invention relates to use of novel pharmaceutical composition in treating or inhibiting a disease condition in a mammal or human in need thereof, comprising administering a therapeutically effective amount of pharmaceutical composition comprising the compound of formula I.

This invention also provides a drug conjugate composition, comprising at least two drug compounds covalently linked to one another via labile bonds to form a single drug conjugate composition.

Another object of the invention is to provide local delivery of two or more synergistic pharmacologic agents or delivery of two or more non-synergistic pharmacologic agents.

In the present invention, when Drug-I, Drug-II and/or Amino Acid linker contain(s) more than one reactive functional group, the undesired functional groups to be protected by conventional methods, before linking them with each other. After the completion of the reactions, the protected groups to be deprotected by conventional methods. For example Drug-I contains an amino and hydroxy groups and the desired group for preparing the conjugates with Drug-II is the amino group. Accordingly, the remaining groups are to be protected before reacting Drug-I with the amino acid linker. Subsequently, the protected groups to be deprotected after preparation of Drug-I and Drug-II conjugates.

The compound described in the present patent application may form salts. Non-limiting examples of pharmaceutically acceptable salts forming part of this patent application include salts derived from inorganic bases salts of organic bases salts of chiral bases, salts of natural amino acids and salts of non-natural amino acids. Certain compounds of present patent application are capable of existing in stereoisomeric forms (e.g. diastereomers and enantiomers). With respect to the overall compounds described by the Formula (I), the present patent application extends to these stereoisomeric forms and to mixtures thereof. To the extent prior art teaches synthesis or separation of particular stereoisomers, the different stereoisomeric forms of the present patent application may be separated from one another by the method known in the art, or a given isomer may be obtained by stereospecific or asymmetric synthesis. Tautomeric forms and mixtures of compounds described herein are also contemplated.

Pharmaceutically acceptable solvates includes hydrates and other solvents of crystallization (such as alcohols). The compounds of the present invention may form solvates with low molecular weight solvents by methods known in the art.

Combination therapy, where in the patient is treated with more than one drug to treat a single disease has become standard of care for many diseases. In particular, metabolic diseases (such as diabetes, obesity), cardiovascular diseases (such as hypertension, dyslipidemia), cancer, HIV-AIDS and other infectious diseases, pain/inflammation and certain CNS diseases are often now treated with combination of two or more drugs. The need to combine drugs is necessitated by the following: a) the disease may be multi-genic and multi-factorial and may involve many mechanisms and to give the best chance of treatment, more than one drug needs to be used b) drug combinations may create therapeutic synergy by affecting multiple disease causing mechanisms, c) combination therapy may use lower doses of the drugs relative to when given singly, therefore decreasing potential side effects and d) combination therapy may result in significant savings due to lower treatment failure rate, lower case-fatality ratios, slower development of resistance and finally less money needed for the development of new drugs.

Despite the many advantages of combining drugs to treat complex diseases, there are also issues that often prevent combining drugs. Chiefly, since the drugs are structurally distinct chemical entities belonging to different chemical classes, they will have inherently discrete physicochemical properties. Physicochemical properties have a strong influence on the absorption, distribution, metabolism and excretion (ADME) properties of each drug. In the most standard situation, a tablet is ingested and passes through the esophagus to the stomach. Because the stomach is an aqueous environment, this is the first place where a tablet will dissolve. However, the physicochemical properties (solubility, partition coefficient, stability and the like) of a drug molecule have a significant effect on its behavior.

Absorption is a primary parameter in the field of pharmacokinetics, since the drug must be absorbed by the gastro-intestinal tract (GI) before any medicinal effects can take place. Moreover, the drug's pharmacokinetic profile can be easily and significantly changed by adjusting factors that affect absorption. The gastrointestinal tract is lined with epithelial cells. Drugs must pass through these cells in order to be absorbed into the circulatory system. One particular cellular barrier that may prevent absorption of a given drug is the cell membrane. Cell membranes are essentially lipid bilayers which form a semi-permeable membrane. Pure lipid bilayers are generally permeable only to small, uncharged solutes. Hence, whether or not a molecule is ionized will affect its absorption, since ionic molecules are considered charged molecules by definition.

Typically a drug is translocated across cell membranes by bulk flow and/or facilitated diffusion. In a combination therapy scenario, since the drugs chemically differ, the transfer by bulk flow can occur by the same mechanism but if the drugs are moving by diffusion their movement is markedly different. The transfer by diffusion of a drug is highly dependent on its solubility in either lipid or water. The rate of dissolution is a key target for controlling the duration of a drug's effect, and as such, several dosage forms that contain the same active ingredient may be available, differing only in the rate of dissolution. If a drug is supplied in a form that is not readily dissolved, the drug may be released more gradually over time with a longer duration of action. The rate of dissolution may be modified primarily by altering the surface area of the solid. The surface area may be adjusted by altering the particle size (e.g. micronization). The rate of dissolution may also be altered by choosing a suitable polymorph of a compound.

Most of the time where combination of drugs is required, the drugs are given as two distinct entities and present as a physical mixture in a formulation. Since the compounds have different physicochemical properties, this often creates a scenario where the absorption of one or the other drug is not optimal and the ADME properties are variable. A classic example of differential physicochemical characteristics is when one drug is a solid and the other drug to be combined is a liquid. In this scenario delivering the two drugs optimally becomes daunting. In combination drug therapy it is important to ensure that optimal concentrations of both drugs are obtained for best therapeutic effects.

In order to overcome poor ADME properties of a drug, a prodrug approach has been used amongst other approaches. A prodrug is a pharmacological substance (drug) that is administered in an inactive (or significantly less active) form. Once administered, the prodrug is metabolized in vivo into an active metabolite. In general, the rationale behind the use of a prodrug is to improve absorption, distribution, metabolism, and excretion and bioavailability. Specifically prodrugs may improve the penetration of a drug across biological membranes in order to obtain improved drug absorption, to prolong duration of action of a drug (slow release of the parent drug from a prodrug, decreased first-pass metabolism of the drug), to target the drug action (e. g. liver or muscle), to improve aqueous solubility and stability of a drug, to improve topical drug delivery (e. g. dermal and ocular drug delivery), to improve the chemical/enzymatic stability of a drug (e. g. peptides) or to decrease drug side-effects.

Many prodrug technologies have been developed and the approach to be used in part depends on the drug that needs improvement (1). Linking of peptides or amino acids to a therapeutic agent has been used as one approach to prodrug. There are at least three examples of the use of amino acids or peptides as prodrugs. One is where the peptide or amino acid represents groups cleavable by specific enzymes that may be present in certain select tissue and therefore serves to target the drug to that tissue. Second is the example of amino acid coupling to a therapeutic compound like valgancyclovir, the L-valyl ester prodrug of gancyclovir, which is used for the prevention and treatment of cytomegalovirus infections (2). After oral administration, valgancyclovir is rapidly converted to gancyclovir by intestinal and hepatic esterases. Alanine and lysine prodrugs of novel antitumor benzothiazoles have been investigated (3). Third example represents peptide carrier-mediated membrane transport of amino acid ester prodrugs of nucleoside analogues. Intestinal epithelial cells express PEPT1, an apical transporter responsible for the uptake of a broad array of small peptides (4). Human PEPT-1 has been found to be the primary absorption pathway of increased systemic delivery of L-valine ester prodrugs. Recently, it was shown that the hPEPT-1 transporter need to optimally interact with a free NH2, a carbonyl group and a lipophylic entity, and may form a few additional H-bridges with its target molecule. L-Valine-linked nucleoside analogue esters may fulfill these requirements for efficient hPEPT-1 substrate activity

It is however important to note that all of these technologies described above are designed with the aim of improving ADME properties of a single drug, not combination of two drugs. There is therefore still a need for new, alternative and better prodrug technologies for application to combination drug therapy. To the best of our knowledge, there is no prior art on using amino-acid linkers to combine two drugs wherein the ester/amide bond is cleaved by esterases/peptidases to release both drugs simultaneously. There is some literature on using acylamino acid linkers to combine HIV drugs, but in those cases, the linker bond is dependent on spontaneous chemical reaction (imide reactions) for drug release (5,6). This presents the problem that if the microenvironment is diverse such as pH in the stomach versus the intestine or circulation, the spontaneous bond cleavage may or may not occur at the intended time, destination or amounts.

The therapeutic drugs each contain a reactive functional group like an amino or hydroxyl or carboxyl group and capable of binding with the carboxyl or amino group of an amino acid via an amide or an ester bond. The amide and ester bonds are cleaved by enzymes such as peptidases and esterases respectively, in the intestine, circulation or liver.

As used herein, the term “pharmaceutically acceptable carrier” includes substances capable of being co-administered with the tetracycline derivatives of general formula (I) and which allow the tetracycline derivatives of general formula (I) to perform their intended function(s), e.g., to treat or prevent bacterial infections. Suitable pharmaceutically acceptable carriers include but are not limited to one or more of water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, colorings, flavorings and/or aromatic substances and the like.

The compounds of the present invention may be administered in a wide variety of different dosage forms. For example, they may be combined with various pharmaceutically acceptable inert carriers in the form of tablets, capsules, lozenges, troches, hard candies, powders, sprays, creams, salves, suppositories, jellies, gels, pastes, lotions, ointments, aqueous suspensions, injectable solutions, elixirs, syrups, and the like, and combinations thereof. Such carriers may include one or more of solid diluents or fillers, sterile aqueous media, and various non-toxic organic solvents, etc. Moreover, oral pharmaceutical compositions may be sweetened and/or flavored. In general, the compounds of the invention may be present in such dosage forms at concentration levels ranging from about 0.1% to about 90% by weight.

The compounds of the present invention may be administered through oral, parenteral or topical routes.

It will be appreciated by those having ordinary skill in the art that the exemplary amounts of active compounds used in a given therapy will vary according to the specific compound being utilized, the particular compositions formulated, the mode of application, the particular site of administration, etc. Optimal administration rates for a given protocol of administration may be ascertained by those having ordinary skill in the art using conventional dosage determination tests conducted with regard to the foregoing guidelines.

The scheme-IV below describes the sequential paradigm for designing drug combinations of the present invention

### REFERENCES

(1) Prodrugs: Challenges and Rewards.
Valentino J. Stella, Ronald T. Borchardt, Michael J. Hageman, Reza Oliyai, Hans Maag, Jefferson Tilley. Published by Springer, 2007.
(2) Antiviral prodrugs—the development of successful prodrug strategies for antiviral chemotherapy;
Erik De Clercq and Hugh J Field. ‘British Journal of Pharmacology (2006) 147, 1-11.
(3) Antitumor benzothiazoles. 16. Synthesis and pharmaceutical properties of antitumor 2-(4-aminophenyl)benzothiazole amino acid prodrugs.
Hutchinson I, Jennings S A, Vishnuvajjala B R, Westwell A D, Stevens M F. J Med Chem. 2002 Jan. 31; 45(3):744.
(4) Human peptide transporters: therapeutic applications
Nielsen C. U.; Brodin B.; Jorgensen F. S.; Frokjaer S.; Steffansen B. Expert Opinion on Therapeutic Patents, Volume 12, Number 9, 1 Sep. 2002, pp. 1329-1350(22).
(5) Development of water-soluble prodrugs of the HIV-1 protease inhibitor KNI-727: importance of the conversion time for higher gastrointestinal absorption of prodrugs based on spontaneous chemical cleavage.
Sohma Y, Hayashi Y, Ito T, Matsumoto H, Kimura T, Kiso Y. J Med Chem. 2003 Sep. 11; 46(19):4124-35.
(6) Design, synthesis, and biological evaluation of anti-HIV double-drugs. conjugates of HIV protease inhibitors with a reverse transcriptase inhibitor through spontaneously cleavable linkers.
Matsumoto H, Kimura T, Hamawaki T, Kumagai A, Goto T, Sano K, Hayashi Y, Kiso Y. Bioorg Med Chem. 2001 June; 9(6):1589-600.

What is claimed is:

1. Compounds represented by formula
Wherein ‘D1’ is Drug-1, which contains one or more functional groups selected from the groups comprising of —OH, —COOH, —SH, —NHR1, —CONH—R1, —SO2NH—R1, —OSO2NH—R1, —NHR1-CO—NHR1, —NR1SO2NH—R1;
R1 represents hydrogen, —NH2, —C(═NH)NH2, alkyl, aryl, heteroaryl, or heterocyclyl;
‘D2’ represents ‘Drug-2’, is selected from a therapeutic, a vitamin, a natural product or a neutraceutical; D2 also contains one or more functional groups as defined above for drug one;
Linker is selected from
i) an amino acid AA
Wherein amino acid AA comprising of two or more functional groups, such as carbohydroxy, hydroxyl, amino, thio or guanidine groups;
Amino acid may be choosen from L- form, D-form or DL-forms;
AA also includes formulas represented by
(a)
wherein R2 is a group selected from (CH2)n-COOH, —CH3, —(CH2)n-NH—C(═NH)—NH2, —CH2SH, —(CH2)nCONH2, —(CH2)n-NH2, —(CH2)nCH2-alkyl, —(CH2)nCH2-heterocylyl, —(CH2)nCH2-heteroaryl, —(CH2)nCH2-aryl, —(CH2)nCH2-phenyl, or —(CH2)nCH2-p-hydroxy phenyl; R2 forms cyclic ring with NH2 as in praline;
(b)NH2-(CH2)n-COOH;
ii) Hydroxy acids represented by
—O—CH(R1)-(CH2)n-COO—
—O—CH(R1)-(CH2)n-CH(R1)-COO—
—O—(CH2)n-CH(R1)-COO—
—OOC—CH(R1)-(CH2)n-O—
—OOC—CH(R1)-(CH2)n-CH(R1)-O—
—OOC—(CH2)n-CH(R1)-O—
—OOC—CH(R1)-(CH2)n-O—
—OOC—(CH2)n-CH(R1)(CH2)n-O—
—O—Ar—CH(R1)-(CH2)n-COO—
—O—Ar—CH(R1)-(CH2)n-CH(R1)-COO—
—O—(CH2)n-Ar—CH(R1)-COO—
—OOC—CH(R1)-Ar—(CH2)n-O—
iii) Mercapto carboxylic acids represented by
—S—CH(R1)-(CH2)n-COO—
—S—CH(R1)-(CH2)n-CH(R1)COO—
—S—(CH2)n-CH(R1)-COO—
—S—(CH2)n-CH(R1)(CH2)n-COO—
—S—Ar—CH(R1)-(CH2)n-O—
—S—CH(R1)-(CH2)n-CH(R1)-Ar—O—
—S—(CH2)n-Ar—CH(R1)-O—
—S—(CH2)n-CH(R1)-Ar—(CH2)n-O—
Iv) Hydroxy mercapto linkers represented by
—O—CH(R1)-(CH2)n-S—
—O—CH(R1)-(CH2)n-CH(R1)-S—
—O—(CH2)n-CH(R1)-S—
—O—(CH2)n-CH(R1)(CH2)n-S—
v) Mercapto hydroxy linkers represented by
—S—CH(R1)-(CH2)n-O—
—S—CH(R1)-(CH2)n-CH(R1)-O—
—S—(CH2)n-CH(R1)-O—
—S—(CH2)n-CH(R1)(CH2)n-O—
vi) Diamino linkers represented by
—NH2-Ar—CH(R1)-(CH2)n-NH2-
—NH2-Ar—CH(R1)-(CH2)n-NH2-
—NH2-CH(R1)-Ar—(CH2)n-CH(R1)-NH2-
—NH2-(CH2)n-CH(R1)-Ar—NH2-
Vii) Dicarboxylic acid linkers represented by
—OOC—CH(R1)-(CH2)n-COO—
—OOC—CH(R1)-(CH2)n-CH(R1)-COO—
—OOC—(CH2)n-CH(R1)-COO—
—O—CH(R1)-(CH2)n-COO—
—OOC—Ar—CH(R1)-(CH2)n-COO—
—OOC—CH(R1)-Ar—(CH2)n-CH(R1)-COO—
Viii) Carboxylicacid mercapto linkers of type
—OOC—(CH2)n-CH(R1)(CH2)n-S—
—O—CO—CH(R1)-(CH2)n-CH(R1)-S—
—O—CO—(CH2)n-CH(R1)-S—
—O—CO—(CH2)n-CH(R1)(CH2)n-S—
—OOC—Ar—(CH2)n-CH(R1)(CH2)n-S—
—O—CO—CH(R1)-(CH2)n-CH(R1)-AR-S—
—O—CO—(CH2)n-Ar—CH(R1)-S—
—O—CO—(CH2)n-CH(R1)-Ar—(CH2)n-S—
ix) Hydroxy amino linkers represented by
—O—CH(R1)-(CH2)n-NH—
—O—CH(R1)-(CH2)n-CH(R1)-NH—
—O—(CH2)n-CH(R1)-NH—
—O—(CH2)n-CH(R1)(CH2)n-NH—
—OAr—CH(R1)-(CH2)n-NH—
—O—CH(R1)-(CH2)n-CH(R1)-Ar—NH—
—O—(CH2)n-Ar—CH(R1)-NH—
—O—(CH2)n-CH(R1)-Ar—(CH2)n-NH—
x) Amino hydroxy linker
—NH—CH(R1)-(CH2)n-OH—
—NH—CH(R1)-(CH2)n-CH(R1)-O—
—NH—(CH2)n-CH(R1)-O—
—NH—(CH2)n-CH(R1)(CH2)n-O—
—NH—CH(R1)-Ar—(CH2)n-OH—
—NH—CH(R1)-(CH2)n-Ar—CH(R1)-O—
—NH—(CH2)n-CH(R1)-Ar—O—
—NH—Ar—(CH2)n-CH(R1)(CH2)n-O—
n is an integer from 0-6;
D1, D2 each independently represents drug of either same therapeutic class or of different therapeutic class; when they represent same therapeutic class each one may be selected from drugs of pharmacologically different mechanism of actions;
D1, D2 are independently represent therapeutically compatible drugs, each independently covalently linked to the two different functional groups of AA linker; AA linker allows the release of D1 or D2 or both in the internal or external environment of the target tissue; D1, D2 may be released either simultaneously or one after the other at different time points, depending on the nature of drug and covalent bond between drug and AA linker and environment of target tissue;
D1 and D2 each independently covalently bonded to Linker through one or more of the following:
v) —O—C(O)—
vi) —S—
vii) —C(O)—NH—
in a more specific embodiment, D1 and D2 each independently is covalently linked either to —NH2 functional group or —COOH functional group of AA;
D1 or D2 is covalently bonded to AA through
i) —O—C(O)—
ii) —NH—C(O)—
iii) —C(O)NH—
iv) —C(O)—O bonds
AA represents an amino acid; which act as linker between Drug-I and Drug-II, selected from the amino acids listed below:
or its stereoisomer thereof.

2. The compound according to claim 1, wherein Drug-I is a drug molecule D1 having carboxyl group(s) as functional group(s) and Drug-II is a drug molecule D2 having hydroxyl group(s) as functional group(s).

3. The compound according to claim 1, wherein Drug-I is a drug molecule D1 having hydroxyl group(s) as functional group(s) and Drug-II is a drug molecule D2 having carboxyl group(s) as functional group(s).

4. The compound according to claim 1, wherein Drug-I and Drug-II represent different therapeutic agents of same or different therapeutic class.

5. The compound according to claim 4, wherein the said therapeutic agents have same or different pharmacological mechanism of actions and/or work on different disease conditions.

6. A pharmaceutical composition comprising a therapeutically effective amount of the compound of claim 1, or a pharmaceutical salt thereof and one or more pharmaceutically acceptable carriers, vehicles or diluents.

7. Use of novel composition of claim 6 in treating a mammal or human in need thereof comprising administering a therapeutically effective amount of pharmaceutical composition comprising the compound of formula (I).

8. A method of developing a compound of formula (I) as provided in claim 1, the said method comprising the following steps:
identifying two known drugs or compounds having potential to become therapeutic agents;
evaluation of amino acid linkers to identify the suitable linker to combine the drugs or compounds identified in step (i);
combining the two drugs or compounds through covalent bond with the linker identified in step (ii), to obtain the compound of formula (I);
in vitro evaluation of the compound formed in step (iii) for intended therapeutic activity;
determining pharmacokinetic experiments in vitro/in vivo to asses the cleavability and to evaluate the release kinetics of the compound of formula (I); and
determining in-vivo efficacy of the compound of formula (I) of step (iii).

9. A method of developing a novel drug conjugate, the said method comprising the following steps:
i. identifying two known drugs or compounds having potential to become therapeutic agents;
ii. evaluation of amino acid linkers to identify the suitable linker to combine the drugs or compounds identified in step (i);
combining the two drugs or compounds through covalent bond with the linker identified in step (ii), to obtain a novel drug conjugate;
in vitro evaluation of the novel drug conjugate formed in step (iii) for intended therapeutic activity;
determining pharmacokinetic experiments in vitro/in vivo to asses the cleavability and to evaluate the release kinetics of combined drugs; and
determining in-vivo efficacy of combined drugs/the novel drug conjugate. novel compounds of formula (I) of step (iii).

10. (canceled)

11. A linker according to claim 1 is amino acid AA comprising of two or more functional groups, such as carboxy, hydroxyl, amino, mercapto or guanidine groups;
Amino acid may be chosen from L- form, D-form or DL-forms;
(i) AA also includes formulas represented by
(a)
wherein R2 is a group selected from (CH2)n-COOH, —CH3, —(CH2)n-NH—C(═NH)—NH2, —CH2SH, —(CH2)nCONH2, —(CH2)n-NH2, —(CH2)nCH2-alkyl, —(CH2)nCH2-heterocylyl, —(CH2)nCH2-heteroaryl, —(CH2)nCH2-aryl, —(CH2)nCH2-phenyl, or —(CH2)nCH2-p-hydroxy phenyl; R2 forms cyclic ring with NH2 as in pro line;
(b)NH2-(CH2)n-COOH;
ii) Hydroxy acids represented by
—O—CH(R1)-(CH2)n-COO—
—O—CH(R1)-(CH2)n-CH(R1)-COO—
—O—(CH2)n-CH(R1)-COO—
—OOC—CH(R1)-(CH2)n-O—
—OOC—CH(R1)-(CH2)n-CH(R1)-O—
—OOC—(CH2)n-CH(R1)-O—
—OOC—CH(R1)-(CH2)n-O—
—OOC—(CH2)n-CH(R1)(CH2)n-O—;
—O—Ar—CH(R1)-(CH2)n-COO—
—O—Ar—CH(R1)-(CH2)n-CH(R1)-COO—
—O—(CH2)n-Ar—CH(R1)-COO—
—OOC—CH(R1)-Ar—(CH2)n-O—
iii) Mercapto carboxylic acids represented by
—S—CH(R1)-(CH2)n-COO—
—S—CH(R1)-(CH2)n-CH(R1)COO—
—S—(CH2)n-CH(R1)-COO—
—S—(CH2)n-CH(R1)(CH2)n-COO—
—S—Ar—CH(R1)-(CH2)n-O—
—S—CH(R1)-(CH2)n-CH(R1)-Ar—O—
—S—(CH2)n-Ar—CH(R1)-O—
—S—(CH2)n-CH(R1)-Ar—(CH2)n-O—
Iv) Hydroxy mercapto linkers represented by
—O—CH(R1)-(CH2)n-S—
—O—CH(R1)-(CH2)n-CH(R1)-S—
—O—(CH2)n-CH(R1)-S—
—O—(CH2)n-CH(R1)(CH2)n-S—
v) Mercapto hydroxy linkers represented by
—S—CH(R1)-(CH2)n-O—
—S—CH(R1)-(CH2)n-CH(R1)-O—
—S—(CH2)n-CH(R1)-O—
—S—(CH2)n-CH(R1)(CH2)n-O—
vi) Diamino linkers represented by
—NH2-Ar—CH(R1)-(CH2)n-NH2-
—NH2-Ar—CH(R1)-(CH2)n-NH2-
—NH2-CH(R1)-Ar—(CH2)n-CH(R1)-NH2-
—NH2-(CH2)n-CH(R1)-Ar—NH2-
Vii) Dicarboxylic acid linkers represented by
—OOC—CH(R1)-(CH2)n-COO—
—OOC—CH(R1)-(CH2)n-CH(R1)-COO—
—OOC—(CH2)n-CH(R1)-COO—
—O—CH(R1)-(CH2)n-COO—
—OOC—Ar—CH(R1)-(CH2)n-COO—
—OOC—CH(R1)-Ar—(CH2)n-CH(R1)-COO—
Viii) Carboxylicacid mercapto linkers of type
—OOC—(CH2)n-CH(R1)(CH2)n-S—
—O—CO—CH(R1)-(CH2)n-CH(R1)-S—
—O—CO—(CH2)n-CH(R1)-S—
—O—CO—(CH2)n-CH(R1)(CH2)n-S—
—OOC—Ar—(CH2)n-CH(R1)(CH2)n-S—
—O—CO—CH(R1)-(CH2)n-CH(R1)-AR-S—
—O—CO—(CH2)n-Ar—CH(R1)-S—
—O—CO—(CH2)n-CH(R1)-Ar—(CH2)n-S—
ix) Hydroxy amino linkers represented by
—O—CH(R1)-(CH2)n-NH—
—O—CH(R1)-(CH2)n-CH(R1)-NH—
—O—(CH2)n-CH(R1)-NH—
—O—(CH2)n-CH(R1)(CH2)n-NH—
—OAr—CH(R1)-(CH2)n-NH—
—O—CH(R1)-(CH2)n-CH(R1)-Ar—NH—
—O—(CH2)n-Ar—CH(R1)-NH—
—O—(CH2)n-CH(R1)-Ar—(CH2)n-NH—
x) Amino hydroxy linker
—NH—CH(R1)-(CH2)n-OH—
—NH—CH(R1)-(CH2)n-CH(R1)-O—
—NH—(CH2)n-CH(R1)-O—
—NH—(CH2)n-CH(R1)(CH2)n-O—
—NH—CH(R1)-Ar—(CH2)n-OH—
—NH—CH(R1)-(CH2)n-Ar—CH(R1)-O—
—NH—(CH2)n-CH(R1)-Ar—O—
—NH—Ar—(CH2)n-CH(R1)(CH2)n-O—
n is an integer from 0-6;
D1, D2 each independently represents drug of either same therapeutic class or of different therapeutic class; when they represent same therapeutic class each one may be selected from drugs of pharmacologically different mechanism of actions;
D1, D2 are independently represent therapeutically compatible drugs, each independently covalently linked to the two different functional groups of AA linker; AA linker allows the release of D1 or D2 or both in the internal or external environment of the target tissue; D1, D2 may be released either simultaneously or one after the other at different time points, depending on the nature of drug and covalent bond between drug and AA linker and environment of target tissue;
D1 and D2 each independently covalently bonded to Linker through one or more of the following:
i) —O—
ii) —C(O)—O—
iii) —NH—
iv) —NH—C(O)—
v) —O—C(O)—
vi) —S—
vii) —C(O)—NH—
in a more specific embodiment, D1 and D2 each independently is covalently linked either to —NH2 functional group or —COOH functional group of AA;
D1 or D2 is covalently bonded to AA through
i) —O—C(O)—
ii) —NH—C(O)—
iii) —C(O)NH—
iv) —C(O)—O bonds
AA represents an amino acid; which act as linker between Drug-I and Drug-II, selected from the amino acids listed below:
or its stereoisomer thereof.
